(19) United States
(12) Patent Application Publication
(10) Pub. No.: US 2020/0164163 A1
(43) Pub. Date: May 28, 2020

(54) NEBULIZER AND FLOW-GUIDING ELEMENT THEREOF

(71) Applicant: HCMed Innovations Co., LTD., Taipei City (TW)

(72) Inventors: Chieh-Sheng CHENG, Taipei City (TW); Wen-Yu TSAI, Taipei City (TW); Chi-Hsueh LIN, Taipei City (TW); Ke-Ting CHEN, Taipei City (TW)

(21) Appl. No.: 16/695,075

(22) Filed: Nov. 25, 2019

Related U.S. Application Data
(60) Provisional application No. 62/771,175, filed on Nov. 26, 2018.

(51) Int. Cl. A61M 15/00; A61M 16/14; A61M 11/00

(52) U.S. Cl. CPC A61M 15/002; A61M 2206/20; A61M 11/005; A61M 16/14

(57) ABSTRACT

A flow-guiding element adapted to a nebulizer having a nebulizing module includes a main flow-guiding body having a main flow-guiding channel, and an auxiliary flow-guiding body having at least one auxiliary flow-guiding channel. The at least one auxiliary flow-guiding channel is communicated with the main flow-guiding channel, and a longitudinal axis of the main flow-guiding body crosses a longitudinal axis of the auxiliary flow-guiding body. A nebulizer includes a nebulizing module and a flow-guiding element connected to the nebulizing module. The flow-guiding element includes a main flow-guiding body having a main flow-guiding channel and an auxiliary flow-guiding body having at least one auxiliary flow-guiding channel, in which the at least one auxiliary flow-guiding channel is communicated with the main flow-guiding channel, and the longitudinal axis of the main flow-guiding body crosses a longitudinal axis of the auxiliary flow-guiding body.

### CROSS-REFERENCE TO RELATED PATENT APPLICATION

This application claims priority from the U.S. Provisional Patent Application Ser. No. 62/771,175 filed Nov. 26, 2018, which application is incorporated herein by reference in its entirety.

Some references, which may include patents, patent applications and various publications, may be cited and discussed in the description of this disclosure. The citation and/or discussion of such references is provided merely to clarify the description of the present disclosure and is not an admission that any such reference is “prior art” to the disclosure described herein. All references cited and discussed in this specification are incorporated herein by reference in their entireties and to the same extent as if each reference was individually incorporated by reference.

### FIELD OF THE DISCLOSURE

The present disclosure relates to a nebulizer and a flow-guiding element, and more particularly to a nebulizer and a flow-guiding element adapted to a nebulizer.

### BACKGROUND OF THE DISCLOSURE

Nebulization therapies for respiratory diseases have gained popularity in recent years for being capable of providing more efficient and more direct therapeutic effects than oral medication.

Nebulizer is used to deliver medication in fine particles to patients for inhalation. A nebulizing module is a component of nebulizer accounting for generating aerosol for treating a patient with respiratory conditions. The nebulizing module typically includes a perforated membrane and a vibratile element. By vibrating the perforated membrane with the vibratile element, the liquid medicament permeating the perforated membrane is dispersed to be aerosol. In the delivery of medication, certain physical properties are required to a drug particle for depositing in the deep lung in order to exert the therapeutic action. Specifically, the drug particle having an aerodynamic diameter below 5 μm is considered as a respirable drug particle; in other words, the aerodynamic diameter of the drug particle must be less than 5 μm when exiting an inhaler, so that the drug particle can deposit in the deep lung. A challenge in effectively providing the respirable drug particle for the patient is that the aerosol can aggregate on the path from the nebulizing module to the outlet of the nebulizer, which will result in a lower respirable fraction of the inhalation drug particles.

### SUMMARY OF THE DISCLOSURE

In response to the above-referenced technical inadequacies, the present disclosure provides a nebulizer and flow-guiding element thereof.

In one aspect, the present disclosure provides a flow-guiding element adapted to a nebulizer having a nebulizing module. The flow-guiding element includes a main flow-guiding body having a main flow-guiding channel and an auxiliary flow-guiding body having at least one auxiliary flow-guiding channel. The at least one auxiliary flow-guiding channel is communicated with the main flow-guiding channel, and a longitudinal axis of the main flow-guiding body crosses a longitudinal axis of the auxiliary flow-guiding body.

In one aspect, the present disclosure provides a nebulizer including a nebulizing module and a flow-guiding element connected to the nebulizing module. The flow-guiding element includes a main flow-guiding body having a main flow-guiding channel and an auxiliary flow-guiding body having at least one auxiliary flow-guiding channel. The at least one auxiliary flow-guiding channel is communicated with the main flow-guiding channel, and a longitudinal axis of the main flow-guiding body crosses a longitudinal axis of the auxiliary flow-guiding body.

Therefore, the flowing of the air in the main flow-guiding channel create a negative pressure in the main flow-guiding channel to enable an environment air to be introduced into the main flow-guiding channel by passing through the auxiliary flow-guiding channel, and the introduced air toward the position of the nebulizing module of the nebulizer, or crosses the airflow and the drug particles in the main flow-guiding channel, so as to disperse drug particles and lower the aggregation degree of the nebulized drug particles.

The nebulizer and the flow-guiding element thereof can effectively increase the drug respirable fraction in the lower respiratory tract of a patient without interfering with the performance of the nebulizer, and in the meantime mitigate the adverse reaction(s) caused by deposition of the drug particles in the mouth or the upper respiratory tract of the patient, thereby greatly increasing the efficiency of a nebulization treatment.

These and other aspects of the present disclosure will become apparent from the following description of the embodiment taken in conjunction with the following drawings and their captions, although variations and modifications therein may be affected without departing from the spirit and scope of the novel concepts of the disclosure.

### BRIEF DESCRIPTION OF THE DRAWINGS

The present disclosure will become more fully understood from the following detailed description and accompanying drawings.

FIG. 1 is a perspective view of a flow-guiding element according to an embodiment of the present disclosure.

FIG. 2 is a perspective view of a flow-guiding element according to one other embodiment of the present disclosure.

FIG. 3 is a perspective view of a flow-guiding element according to one other embodiment of the present disclosure.

FIG. 4 is a schematic view of a flow-guiding element connected with a nebulizer according to one other embodiment of the present disclosure.

FIG. 5 is a perspective view of a mouthpiece combined with a flow-guiding element according to an embodiment of the present disclosure.

FIG. 6 is a perspective view of a mouthpiece combined with a flow-guiding element according to one other embodiment of the present disclosure.

FIG. 7 is a perspective view of a mouthpiece combined with a flow-guiding element according to one other embodiment of the present disclosure.

FIG. 8 is a perspective view of a mouthpiece integrally formed with a flow-guiding element according to an embodiment of the present disclosure.

FIG. 9 is a perspective view of a mouthpiece integrally formed with a flow-guiding element according to one other embodiment of the present disclosure.

FIG. 10 is a perspective view of a mouthpiece integrally formed with a flow-guiding element according to one other embodiment of the present disclosure.

FIG. 11 is a perspective view of a flow-guiding element according to one other embodiment of the present disclosure.

FIG. 12 is a schematic view of a flow-guiding element connected with a nebulizer according to one other embodiment of the present disclosure.

FIG. 13 is a perspective view of a flow-guiding element according to one other embodiment of the present disclosure.

FIG. 14 is a schematic view of a flow-guiding element connected with a nebulizer according to one other embodiment of the present disclosure.

FIG. 15 is a schematic view of a flow-guiding element connected with a nebulizer according to one other embodiment of the present disclosure.

### DETAILED DESCRIPTION OF THE EXEMPLARY EMBODIMENTS

The following examples are intended as illustrative only since numerous modifications and variations therein will be apparent to those skilled in the art.

Like numbers in the drawings indicate like components throughout the views. As used in the description herein and throughout the claims that follow, unless the context clearly dictates otherwise, the meaning of “a”, “an”, and “the” includes plural reference, and the meaning of “in” includes “in” and “on”. Titles or subtitles can be used herein for the convenience of a reader, which shall have no influence on the scope of the present disclosure.

The terms used herein generally have their ordinary meanings in the art. In the case of conflict, the present document, including any definitions given herein, will prevail. The same thing can be expressed in more than one way. Alternative language and synonyms can be used for any term(s) discussed herein, and no special significance is to be placed upon whether a term is elaborated or discussed herein. A recital of one or more synonyms does not exclude the use of other synonyms. The use of examples anywhere in this specification including examples of any terms is illustrative only, and in no way limits the scope and meaning of the present disclosure or of any exemplified term. Likewise, the present disclosure is not limited to various embodiments given herein. Numbering terms such as “first”, “second” or “third” can be used to describe various components, signals or the like, which are for distinguishing one component/signal from another one only, and are not intended to, nor should be construed to impose any substantive limitations on the components, signals or the like.

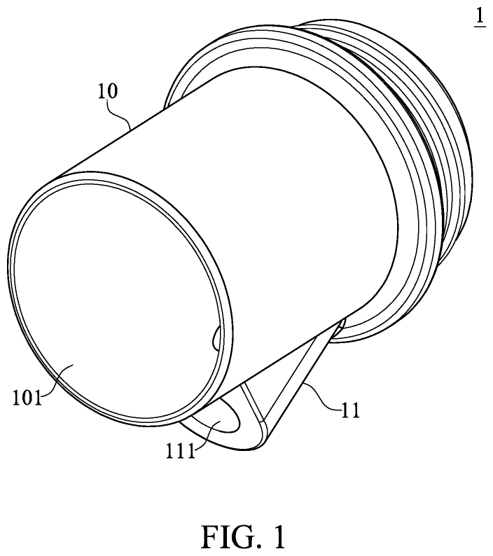

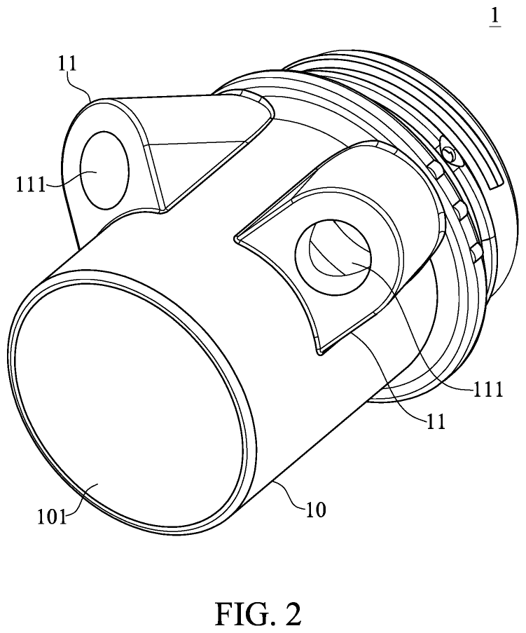

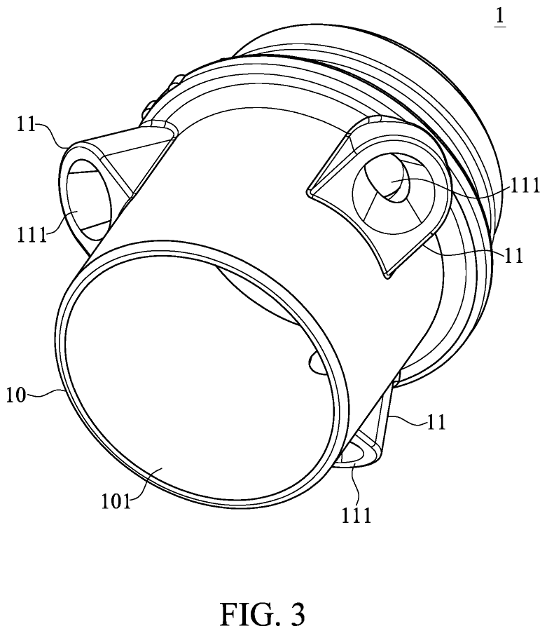

As shown in FIGS. 1-4, an auxiliary flow-guiding body 11 is provided on a main flow-guiding body 10 for a nebulizer 2 to increase the respirable fraction of inhalation drug for a patient, and lower the percentage of the coarse nebulized drug particles that deposit in the mouth or upper respiratory tract of the patient and the likelihood of the adverse reaction(s) caused by such drug deposition. Specifically, drug particles DP nebulized by a nebulizing module 21 of the nebulizer 2 may enter a main flow-guiding channel 101 of the main flow-guiding body 10 connected to the nebulizer 2 and be carried by the airflow therein in a longitudinal direction of the main flow-guiding body 10 to flow along a first flowing path P1 and away from the nebulizer 2. The flowing of the air in the main flow-guiding channel 101 create a negative pressure in the main flow-guiding channel 101 to enable an environment air A to be introduced into the main flow-guiding channel 101 by passing through the auxiliary flow-guiding body 11, and a second flowing path P2 of the introduced air toward the position of the nebulizing module 21 of the nebulizer 2, or crosses first flowing path P1 of the airflow and the drug particles DP in the main flow-guiding channel 101, so as to further disperse drug particles DP and lower the aggregation degree of the nebulized drug particles DP.

In certain embodiments, the auxiliary flow-guiding body 11 can include at least one auxiliary flow-guiding channel.

In certain embodiments, the at least one auxiliary flow-guiding channel can be one oblique flow-guiding channel 111 or a plurality of oblique flow-guiding channels 111, and a longitudinal axis of the auxiliary flow-guiding body 11 crosses the nebulizing module 21 of the nebulizer 2. The shape and contour of the auxiliary flow-guiding body 11 are not limited in the present disclosure. As shown below in Table 1, which presents a comparison between a flow-guiding element 1 provided with no oblique flow-guiding channel and two embodiments of a flow-guiding element 1 provided with at least one oblique flow-guiding channel 111 according to the present disclosure, when the aerosol output rate remains relatively unaffected, a main flow-guiding body 10 provided with the oblique flow-guiding channel(s) 111 effectively decreases the mass median aerodynamic diameter (MMAD) of the nebulized drug particles by 17% to 27%, and increases the fine particle fraction (FPF) of the nebulized drug particles by 23% to 40%. It is noted that Aerosol output rate (g/min) refers to the rate of nebulization and the rate of drug delivery, MMAD (μm) is a parameter of the diameter of the nebulized drug particles, FPF (%) refers to a fraction of the drug particles whose diameter are smaller than 5 μm after nebulization, and those drug particles are considered as respirable drug particles.

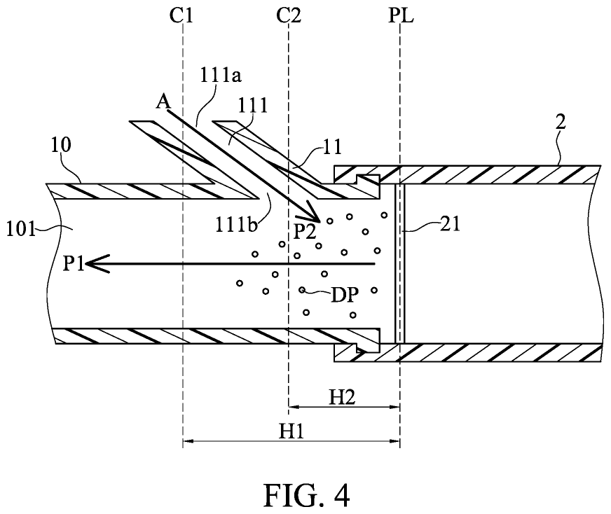

As shown in FIGS. 1-3, the main flow-guiding body 10 for the nebulizer 2 is provided with the auxiliary flow-guiding body 11 including one, two or three oblique flow-guiding channel(s) 111, so as to increase the respirable fraction of the inhalation drug particles DP for a patient. As shown in FIG. 4, each of the oblique flow-guiding channels 111 has a first opening 111a formed on the outer surface of the auxiliary flow-guiding body 11, and a second opening 111b formed on the inner surface of the main flow-guiding body 10. The longitudinal shape of the oblique flow-guiding channel(s) 111 serves to guide and unify the flowing directions of the air introduced from the first opening 111a into the oblique flow-guiding channel(s) 111. When the main flow-guiding body 10 is combined with the nebulizer 2, a first horizontal distance H1 between a center C1 of the first opening 111a and a plane PL where the nebulizing module 21 of the nebulizer 2 is located is greater than a second horizontal distance H2 between a center C2 of the second opening 111b and the plane PL. A longitudinal axis of each of the oblique flow-guiding channel(s) 111 crosses the position of the nebulizing module 21 in the nebulizer 2, and crosses a portion of the first flowing path P1 of the nebulized drug particles DP in the main flow-guiding channel 101 that is between the nebulizing module 21 and a vertical projection of the second opening 111b on the first flowing path P1. In certain embodiments, the nebulizing module 21 can be a nebulizing sheet that nebulizes drug particles when operating in coordination with a vibrating sheet. The nebulized drug particles DP may be generated from only a nebulizing portion of the nebulizing module 21, for example, a central portion of the surface thereof that faces the main flow-guiding body 10 when the main flow-guiding body 10 is combined with the nebulizer 2, instead of being generated from the entire surface thereof. The longitudinal axis of the oblique flow-guiding channel(s) 111 may be arranged to cross the position of the nebulizing portion of the nebulizing module 21.

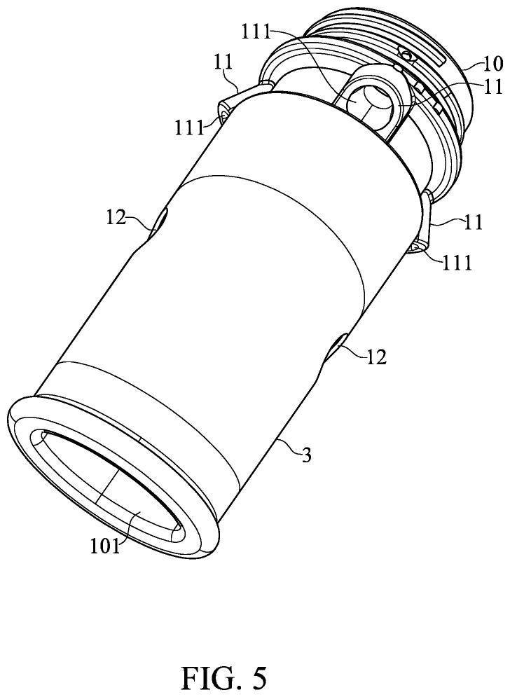

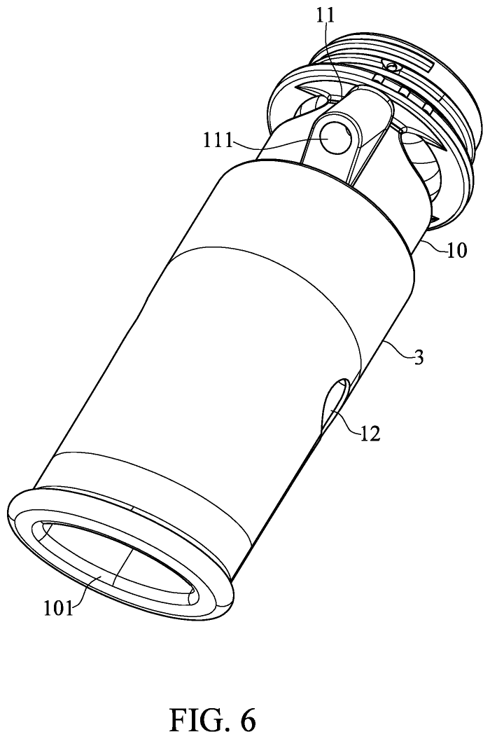

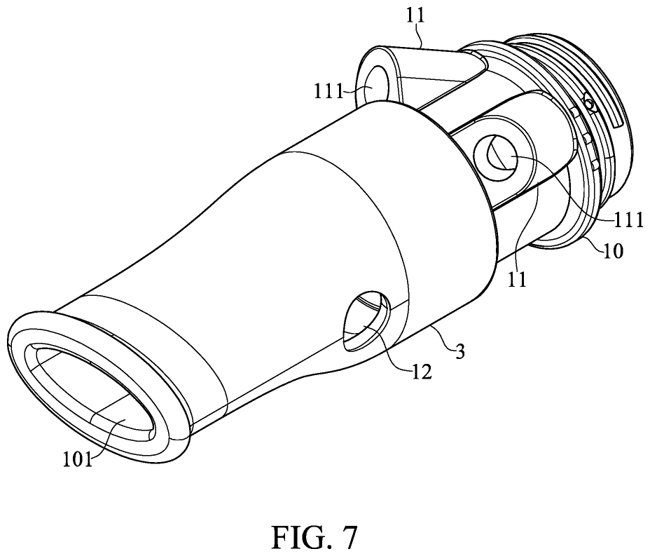

As shown in FIGS. 5-7, a mouthpiece 3 may be combined with the main flow-guiding body 10 provided with the auxiliary flow-guiding body 11 including one, two or three oblique flow-guiding channel(s) 111 without hindering the air path(s) from the outside of the auxiliary flow-guiding body 11 into the oblique flow-guiding channel(s) 111.

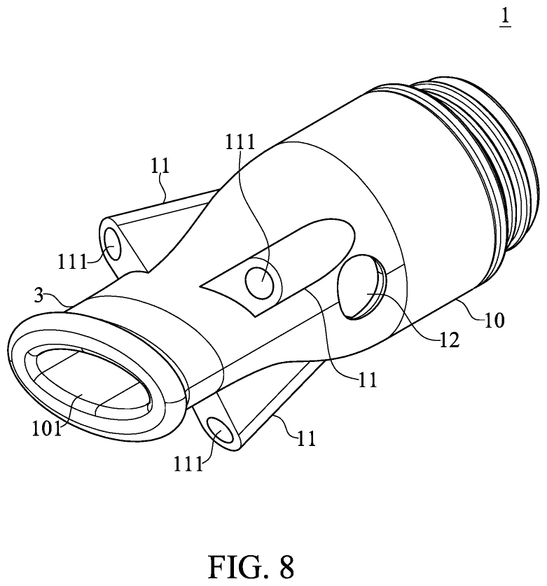

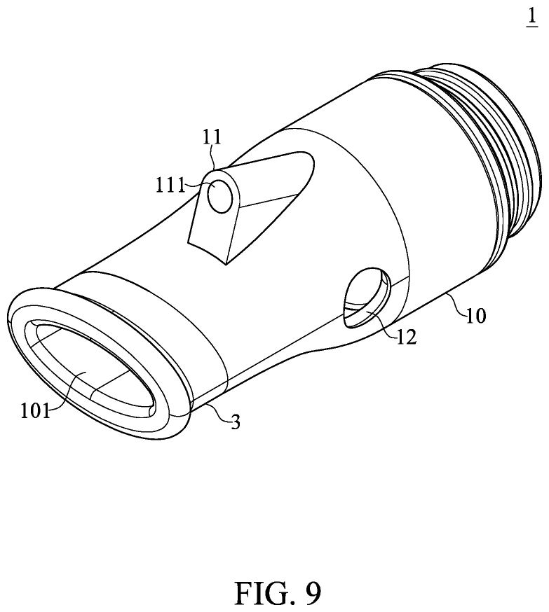

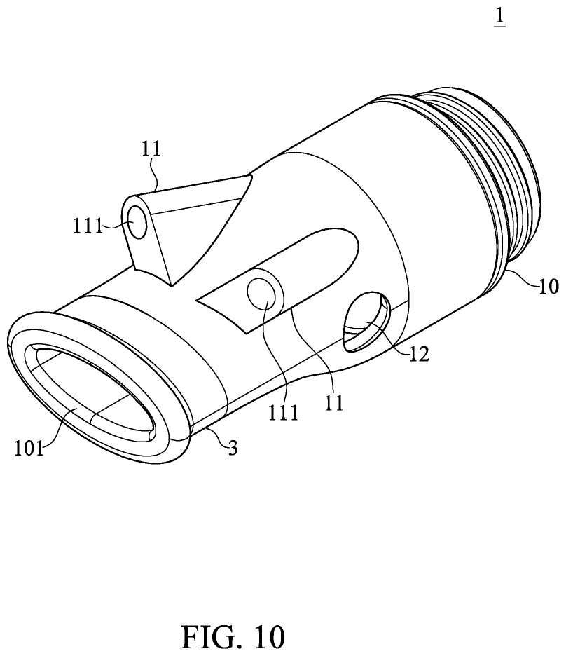

In certain embodiments, as shown in FIGS. 8-10, the mouthpiece 3 may be integrally formed with the main flow-guiding body 10 for the nebulizer 2 and be provided with the auxiliary flow-guiding body 11 including one, two or three oblique flow-guiding channel(s) 111, so as to increase the respirable fraction of the nebulized drug particles DP.

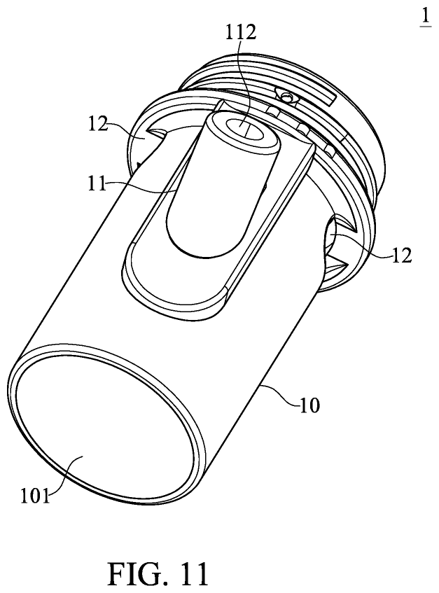

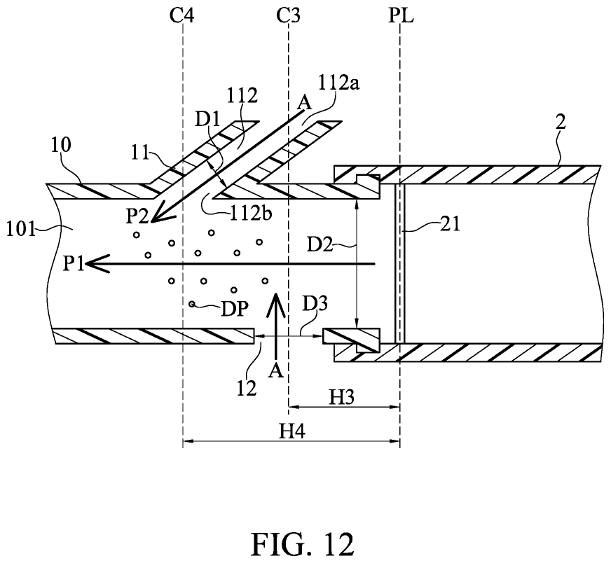

In certain embodiments, as shown in FIG. 11 and FIG. 12, the main flow-guiding body 10 is provided with the auxiliary flow-guiding body 11 including at least one reverse oblique flow-guiding channel 112. As shown in FIG. 12, the reverse oblique flow-guiding channel 112 has a first opening 112a formed on the outer surface of the auxiliary flow-guiding body 11, and a second opening 112b formed on the inner surface of the main flow-guiding body 10. When the main flow-guiding body 10 is combined with the nebulizer 2, a third horizontal distance H3 between a center C3 of the first opening 112a and the plane PL is smaller than a fourth horizontal distance H4 between a center C4 of the second opening 112b and the plane PL. A longitudinal axis of the reverse oblique flow-guiding channel 112 crosses a portion of the first flowing path P1 of the nebulized drug particles DP in the main flow-guiding channel 101 that is between the vertical projection of the second opening 112b of the reverse oblique flow-guiding channel 112 on the first flowing path P1 and an end of the main flow-guiding body 10 that is opposite to a nebulizer-connectable end of the main flow-guiding body 10.

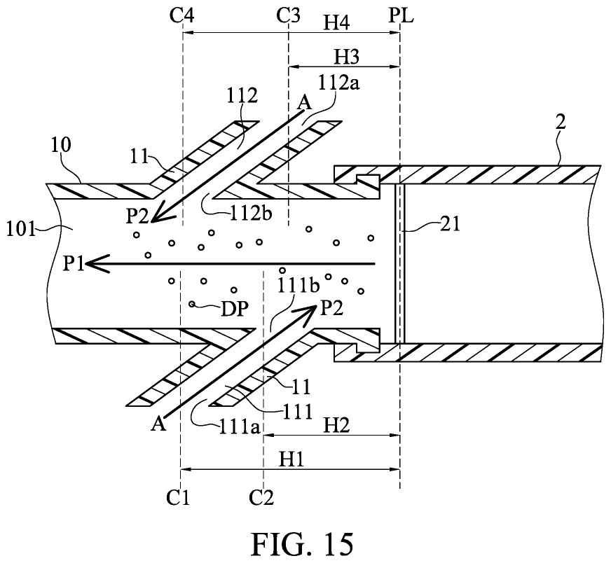

In certain embodiments, the main flow-guiding body 10 provided with the at least one auxiliary flow-guiding body 11 including the oblique flow-guiding channel 111 and the reverse oblique flow-guiding channel 112. As shown in FIG. 15, the environment air A is introduced from both the oblique flow-guiding channel 111 and the reverse oblique flow-guiding channel 112, so the environment air A flows across the first flowing path P1 and further disperses nebulized drug particles DP, therefore to lower the aggregation degree of the nebulized drug particles DP.

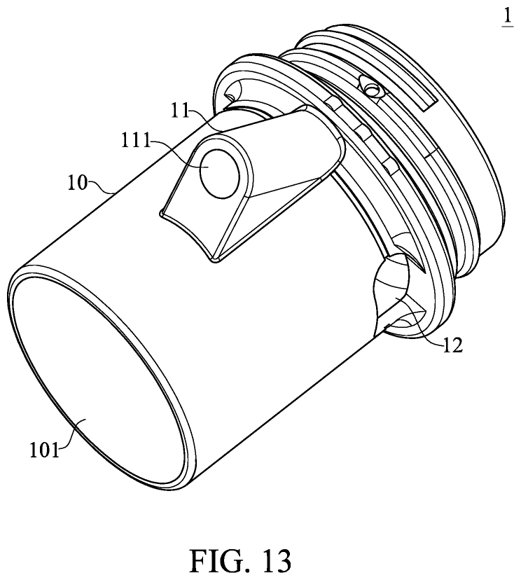

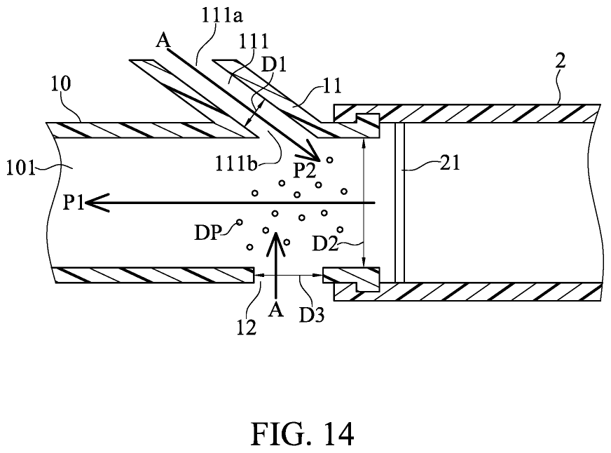

In certain embodiments, as shown in FIGS. 5-14, the main flow-guiding body 10 provided with the auxiliary flow-guiding body 11 including at least one oblique flow-guiding channel 111 or the reverse oblique flow-guiding channel 112 is further formed with at least one lateral hole 12. The lateral holes 12 are formed on and penetrate the outer surface of the main flow-guiding body 10, and are communicated with the main flow-guiding channel 101 inside of the main flow-guiding body 10, so as to facilitate the oblique flow-guiding channel(s) 111 or the reverse oblique flow-guiding channel(s) 112 to further disperse the nebulized drug particles DP. Specifically, the flowing of the airflow in the main flow-guiding channel 101 away from the nebulizer 2 along the first flowing path P1 creates a negative pressure in the main flow-guiding channel 101 to enable the environment air A to be further introduced into the main flow-guiding channel 101 by passing through the lateral hole 12 in addition to flowing along the second flowing path P2 into the oblique flow-guiding channel(s) 111 or the reverse oblique flow-guiding channel(s) 112 and then into the main flow-guiding channel 101. The environment air A introduced from the lateral hole 12 flows across the first flowing path P1 and further disperses nebulized drug particles DP so as to lower the aggregation degree of the nebulized drug particles DP.

In certain embodiments, a diameter D1 of the auxiliary flow-guiding channel is smaller than a diameter D2 of the main flow-guiding channel 101. With a smaller cross-sectional area of the auxiliary flow-guiding channel and a larger cross-sectional area of the main flow-guiding channel 101, the flowing of the airflow in the auxiliary flow-guiding channel is faster than the airflow in the main flow-guiding channel 101, so as to improve the effect of dispersing nebulized drug particles DP.

In certain embodiments, a diameter D3 of the lateral hole 12 is larger than the diameter D1 of the auxiliary flow-guiding channel with a larger cross-sectional area of the lateral hole 12 and the smaller cross-sectional area of the auxiliary flow-guiding channel, the negative pressure created by the airflow in the main flow-guiding channel 101 can introduce sufficient airflows from both the environment air A from both the lateral hole 12 and the auxiliary flow-guiding channel, so as to further improve the effect of dispersing nebulized drug particles DP.

In certain embodiments, instead of the oblique flow-guiding channel(s) 111 or the reverse oblique flow-guiding channel(s) 112, the main flow-guiding body 10 may be provided with the auxiliary flow-guiding body 11 including at least one vertical flow-guiding channel that forms an included angle of 90 degrees with the outer surface of the main flow-guiding body 10. The vertical flow-guiding channel has a first opening formed on the outer surface of the auxiliary flow-guiding body 11, and a second opening formed on the inner surface of the main flow-guiding body 10. The vertical flow-guiding channel has a longitudinal shape that serves to guide and unify the flowing directions of the air inside of the vertical flow-guiding channel that is introduced from the first opening because of the negative pressure in the main flow-guiding channel 101. The longitudinal axis of the vertical flow-guiding channel crosses the first flowing path P1 of the nebulized drug particles DP inside of the main flow-guiding channel 101.

Accordingly, the flow-guiding element 1 provided with the auxiliary flow-guiding body 11 including at least one oblique flow-guiding channel 111, the reverse oblique flow-guiding channel 112, the vertical low-guiding channel, or the combination thereof can effectively increase the drug respirable fraction in the lower respiratory tract of a patient without interfering with the performance of the nebulizer 2, and in the meantime mitigate the adverse reaction(s) caused by deposition of the drug particles in the mouth or the upper respiratory tract of the patient, thereby greatly increasing the efficiency of a nebulization treatment.

The foregoing description of the exemplary embodiments of the present disclosure has been presented only for the purposes of illustration and description and is not intended to be exhaustive or to limit the present disclosure to the precise forms disclosed. Many modifications and variations are possible in light of the above teaching.

Certain embodiments were chosen and described in order to explain the principles of the present disclosure and their practical application so as to enable others skilled in the art to utilize the present disclosure and various embodiments and with various modifications as are suited to the particular use contemplated. Alternative embodiments will become apparent to those skilled in the art to which the present disclosure pertains without departing from its spirit and scope.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope.

What is claimed is:

1. A flow-guiding element adapted to a nebulizer having a nebulizing module, comprising:
a main flow-guiding body having a main flow-guiding channel; and
an auxiliary flow-guiding body having at least one auxiliary flow-guiding channel;
wherein the at least one auxiliary flow-guiding channel is communicated with the main flow-guiding channel;
wherein a longitudinal axis of the main flow-guiding body crosses a longitudinal axis of the auxiliary flow-guiding body.

2. The flow-guiding element according to claim 1, wherein the at least one auxiliary flow-guiding channel has a first opening formed on an end of the auxiliary flow-guiding body and a second opening formed on another end of the auxiliary flow-guiding body connected with the main flow-guiding body, wherein the at least one auxiliary flow-guiding channel has a longitudinal shape for guiding and unifying a number of flowing directions of an environment air introduced from the first opening.

3. The flow-guiding element according to claim 2, wherein the main flow-guiding channel has a first flowing path for guiding a first airflow with a plurality of drug particles inside the main flow-guiding channel, and a second flowing path for guiding a second airflow introduced from the first opening.

4. The flow-guiding element according to claim 3, wherein the auxiliary flow-guiding channel is an oblique flow-guiding channel relative to the main flow-guiding channel, a reverse oblique flow-guiding channel relative to the main flow-guiding channel, or a vertical flow-guiding channel relative to the main flow-guiding channel.

5. The flow-guiding element according to claim 4, wherein when the flow-guiding element is connected with the nebulizer, the auxiliary flow-guiding channel has a first horizontal distance between a center of the first opening and a plane where the nebulizing module is located that is greater than a second horizontal distance between a center of the second opening and the plane.

6. The flow-guiding element according to claim 5, wherein the longitudinal axis of the flow-guiding channel crosses the nebulizing module.

7. The flow-guiding element according to claim 4, wherein when the flow-guiding element is connected with the nebulizer, the flow-guiding channel has a third horizontal distance between a center of the first opening and a plane where the nebulizing module is located that is smaller than a fourth horizontal distance between a center of the second opening and the plane.

8. The flow-guiding element according to claim 4, wherein the vertical flow-guiding channel has an included angle of 90 degrees relative to the main flow-guiding channel.

9. The flow-guiding element according to claim 4, wherein the main flow-guiding body is connected with a mouthpiece.

10. The flow-guiding element according to claim 1, further comprising at least one lateral hole passing through the main flow-guiding body and communicated with the main flow-guiding channel, wherein a diameter of the at least one auxiliary flow-guiding channel is smaller than a diameter of the main flow-guiding channel, and a diameter of the at least one lateral hole is larger than the diameter of the at least one auxiliary flow-guiding channel.

11. A nebulizer comprising a nebulizing module and a flow-guiding element connected to the nebulizing module, the flow-guiding element comprising:
a main flow-guiding body having a main flow-guiding channel; and
an auxiliary flow-guiding body having at least one auxiliary flow-guiding channel;
wherein the at least one auxiliary flow-guiding channel is communicated with the main flow-guiding channel;
wherein a longitudinal axis of the main flow-guiding body crosses a longitudinal axis of the auxiliary flow-guiding body.

12. The nebulizer according to claim 11, wherein the at least one auxiliary flow-guiding channel has a first opening formed on an end of the auxiliary flow-guiding body and a second opening formed on another end of the auxiliary flow-guiding body connected with the main flow-guiding body, wherein the at least one auxiliary flow-guiding channel has a longitudinal shape for guiding and unifying a number of flowing directions of an environment air introduced from the first opening.

13. The nebulizer according to claim 12, wherein the main flow-guiding channel has a first flowing path for guiding a first airflow with a plurality of drug particles inside the main flow-guiding channel, and a second flowing path for guiding a second airflow introduced from the first opening.

14. The nebulizer according to claim 13, wherein the auxiliary flow-guiding channel is an oblique flow-guiding channel relative to the main flow-guiding channel, a reverse oblique flow-guiding channel relative to the main flow-guiding channel, or a vertical flow-guiding channel relative to the main flow-guiding channel.

15. The nebulizer according to claim 14, wherein when the flow-guiding element is connected with the nebulizer, the flow-guiding channel has a first horizontal distance between a center of the first opening and a plane where the nebulizing module is located that is greater than a second horizontal distance between a center of the second opening and the plane.

16. The nebulizer according to claim 15, wherein the longitudinal axis of the flow-guiding channel crosses the nebulizing module.

17. The nebulizer according to claim 14, wherein when the flow-guiding element is connected with the nebulizer, the flow-guiding channel has a third horizontal distance between a center of the first opening and a plane where the nebulizing module is located that is smaller than a fourth horizontal distance between a center of the second opening and the plane.

18. The nebulizer according to claim 14, wherein the vertical flow-guiding channel has an included angle of 90 degrees relative to the main flow-guiding channel.

19. The nebulizer according to claim 14, wherein the main flow-guiding body is connected with a mouthpiece.

20. The nebulizer according to claim 11, further comprising at least one lateral hole passing through the main flow-guiding body and communicated with the main flow-guiding channel, wherein a diameter of the at least one auxiliary flow-guiding channel is smaller than a diameter of the main flow-guiding channel, and a diameter of the at least one lateral hole is larger than the diameter of the at least one auxiliary flow-guiding channel.
